(19) United States
(12) Patent Application Publication
(10) Pub. No.: US 2021/0063977 A1
(43) Pub. Date: Mar. 4, 2021

(54) INFORMATION PROCESSING APPARATUS AND NON-TRANSITORY COMPUTER READABLE MEDIUM STORING PROGRAM

(71) Applicant: FUJI XEROX CO., LTD., Tokyo (JP)

(72) Inventors: Masahiro SATO, Kanagawa (JP); Kengo TOKUCHI, Kanagawa (JP)

(73) Assignee: FUJI XEROX CO., LTD., Tokyo (JP)

(21) Appl. No.: 16/741,748

(22) Filed: Jan. 14, 2020

(30) Foreign Application Priority Data
Aug. 26, 2019 (JP) 2019-153631

(51) Int. Cl. G05B 15/02; G16H 50/30

(52) U.S. Cl. CPC G05B 15/02; A61B 5/165; G16H 50/30

(57) ABSTRACT

An information processing apparatus includes a processor configured to receive biological information of a user, and output, to a device which implements means for coping with a state of the user recognized from the biological information, an operation instruction for implementing the means.

### CROSS-REFERENCE TO RELATED APPLICATIONS

This application is based on and claims priority under 35 USC 119 from Japanese Patent Application No. 2019-153631 filed Aug. 26, 2019.

### BACKGROUND

### (i) Technical Field

The present invention relates to an information processing apparatus and a non-transitory computer readable medium storing a program.

### (ii) Related Art

It is conceivable to operate a device using biological information such as brain waves.

JP2015-211705A describes an apparatus that detects an electroencephalographic signal from user's brain waves, detects a surface myoelectric biological signal from the user's surface myoelectric potential, and calculates a control signal based on both biological signals.

JP2016-067922A describes a device that acquires a user's brain wave, and selectively operates a plurality of operation devices according to an analysis result obtained by analyzing the brain wave.

### SUMMARY

However, in a case where there is a plurality of means capable of coping with the state of the user, it may be difficult to know which device to be given an operation instruction in order to implement the means.

Aspects of non-limiting embodiments of the present disclosure relate to an information processing apparatus and a non-transitory computer readable medium storing a program, which output an operation instruction to a device capable of coping with the state of a user.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus including a processor configured to receive biological information of a user, and output, to a device which implements means for coping with a state of the user recognized from the biological information, an operation instruction for implementing the means.

### BRIEF DESCRIPTION OF THE DRAWINGS

Exemplary embodiment(s) of the present invention will be described in detail based on the following figures, wherein:

FIG. 1 is a block diagram illustrating a configuration of an information processing system according to the present exemplary embodiment;

FIG. 2 is a block diagram illustrating a configuration of an information processing apparatus according to the present exemplary embodiment;

FIG. 3 is a flowchart showing a process by the information processing apparatus according to the present exemplary embodiment;

FIG. 4 is a diagram illustrating a management table;

FIG. 5 is a diagram illustrating a management table; and

FIG. 6 is a diagram illustrating a management table.

### DETAILED DESCRIPTION

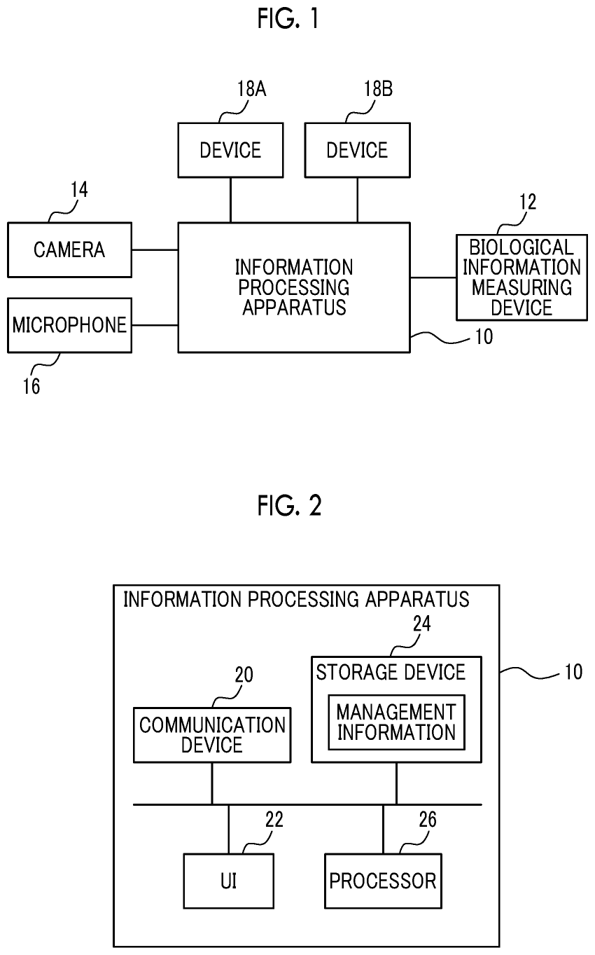

An information processing system according to the present exemplary embodiment will be described with reference to FIG. 1. FIG. 1 illustrates an example of the configuration of the information processing system according to the present exemplary embodiment.

The information processing system according to the present exemplary embodiment includes an information processing apparatus 10, one or a plurality of biological information measuring devices 12, one or a plurality of cameras 14, one or a plurality of microphones 16, and one or a plurality of devices. In the example shown in FIG. 1, the information processing system includes devices 18A, 18B, but this is only an example. Hereinafter, in a case where it is not necessary to distinguish the devices 18A, 18B, the devices 18A, 18B are referred to as “device 18”. The number of devices and sensors shown in FIG. 1 is only an example, and the number of devices and sensors is not limited to the number of devices and sensors shown in FIG. 1. The information processing system may include other devices (for example, external devices such as servers) other than the device shown in FIG. 1.

The information processing apparatus 10, the biological information measuring device 12, the camera 14, the microphone 16, and the device 18 are configured to communicate with other devices, other sensors, and the like. The communication may be wired communication using a cable, or wireless communication. That is, each device and each sensor may be physically connected to another device, another sensor, or the like by a cable to transmit and receive information to and from each other, or may transmit and receive information to and from each other by wireless communication. For example, near field communication, Wi-Fi (registered trademark), or the like is used as the wireless communication. Wireless communication of standards other than these may be used. Near field communication is, for example, Bluetooth (registered trademark), radio frequency identifier (RFID), NFC, or the like. Each device may communicate with another device, another sensor, or the like through a communication path such as a local area network (LAN) or the Internet.

The information processing apparatus 10 is, for example, a personal computer (hereinafter referred to as “PC”), a tablet PC, a smartphone, a mobile phone, or other apparatuses. The information processing apparatus 10 may be a terminal apparatus (for example, a tablet PC, a smartphone, a mobile phone, or the like) that can be carried by the user, or may be a device that is installed on a table or the like and used. The information processing apparatus 10 may be a smart speaker having a communication function, a microphone, and a speaker. The information processing apparatus 10 may be installed indoors (for example, a room floor, a ceiling, a table, or the like) or may be installed outdoors. Further, the information processing apparatus 10 may be a movable apparatus (for example, a self-propelled apparatus).

The biological information measuring device 12 includes a sensor, an electrode, and the like, and is configured to measure a user's biological information. In a case where the plurality of biological information measuring devices 12 are included in the information processing system, biological information measuring devices 12 may measure different types of biological information, respectively. Of course, a part or all of the biological information measuring devices 12 may be configured to measure the same type of biological information. Each biological information measuring device 12 may be configured to measure one type of biological information or may be configured to measure a plurality of types of biological information.

The biological information measuring device 12 transmits the biological information measured by the own device to the information processing apparatus 10. The biological information measuring device 12 may transmit the biological information to the information processing apparatus 10 every time the biological information is measured, or store the biological information and transmit the biological information to the information processing apparatus 10 at predetermined time intervals, or transmit the biological information to the information processing apparatus 10 at a timing designated by the user. The biological information measuring device 12 may receive the biological information measured by another type of biological information measuring device 12 from the other biological information measuring device 12, and transmit the biological information measured by the own device and the biological information measured by the other type of biological information measuring device 12 to the information processing apparatus 10.

The biological information measuring device 12 may analyze the biological information measured by the own device or the other type of biological information measuring device, and transmit information indicating the analysis result to the information processing apparatus 10. For example, the biological information measuring device 12 may include a processor, and the processor may analyze the biological information. Of course, the analysis may be performed by the information processing apparatus 10.

The biological information measuring device 12 includes a battery, and may be driven by power supplied from the battery, or may be driven by receiving power supplied from the information processing apparatus 10.

The biological information measuring device 12 may be a wearable device that measures biological information by the entire biological information measuring device 12 being worn on a user. For example, the biological information measuring device 12 may be a device worn on the user's head, a bearable device worn on the user's ear, or a device worn on the user's arm, hand, wrist, or finger (for example, a wristwatch-type device), a device worn around the user's neck, or a device worn on the user's body or legs.

The biological information is various types of physiological information and anatomical information emitted from a user who is a living body. The category of the concept of biological information includes, for example, information indicating brain activity (for example, brain wave, cerebral blood flow, cerebral magnetic field signal, or the like), pulse rate, blood pressure, heart rate, electrocardiographic waveform, electromyographic waveform, eye movement, body temperature, sweating, line of sight, voice, user's movement and the like. These are only examples of biological information, and other types of physiological information or anatomical information may be used as the biological information. The biological information measuring device 12 may measure one piece of biological information among these pieces of biological information, or may measure a plurality of pieces of biological information.

The information processing apparatus 10 receives biological information from the biological information measuring device 12, and analyzes the biological information, stores the biological information, outputs the biological information, stores information indicating the analysis result of the biological information, and outputs information indicating the analysis result of the biological information. Of course, analysis of biological information may be performed by the biological information measuring device 12. Outputting the biological information includes, for example, displaying the biological information, outputting the biological information as voice information, and the like. Outputting information indicating the analysis result of the biological information includes, for example, displaying information indicating the analysis result, outputting the analysis result as voice information, and the like. The information processing apparatus 10 may transmit biological information and information indicating the analysis result to another apparatus.

The information processing apparatus 10 may include one or a plurality of biological information measuring devices 12. That is, one or a plurality of biological information measuring devices 12 may be incorporated into the information processing apparatus 10 to constitute one device. The entire information processing apparatus 10 including one or a plurality of biological information measuring devices 12 may be worn by a user to measure biological information. That is, the information processing apparatus 10 may be a wearable apparatus. For example, the information processing apparatus 10 may be a device worn on the user's head, a bearable device worn on the user's ear, or a device worn on the user's arm, hand, wrist, or finger (for example, a wristwatch-type device), a device worn around the user's neck, or a device worn on the user's body or legs.

Of course, the information processing apparatus 10 and the biological information measuring device 12 may be separate devices. For example, the information processing apparatus 10 may be a smart speaker, and the biological information measuring device 12 may be a wearable device worn by a user.

The camera 14 is an imaging device. The surroundings of the information processing apparatus 10 and other places are captured by the camera 14, and image data representing the surroundings and image data representing other places are generated. The image data may be moving image data or still image data. The image data captured by the camera 14 corresponds to an example of environment information indicating an environment included in the capturing range of the camera 14. In that sense, the camera 14 corresponds to an example of an environment information measuring device. Further, the image data representing the user, generated by capturing the user with the camera 14, corresponds to an example of the biological information of the user. For example, the movement of the user, the body shape of the user, and the like detected from the image data correspond to an example of the biological information of the user. In that sense, the camera 14 corresponds to an example of a biological information measuring device that measures biological information of the user.

The microphone 16 is a device that collects sound waves. Sound around the microphone 16 (for example, human voice or other sounds) is input to the microphone 16, and sound data is generated by the microphone 16. The sound data representing the sound input to the microphone 16 corresponds to an example of environment information indicating the environment around the microphone 16. In that sense, the microphone 16 corresponds to an example of an environment information measuring device. The sound data representing the user's voice input to the microphone 16 corresponds to an example of the user's biological information. In that sense, the microphone 16 corresponds to an example of a biological information measuring device that measures biological information of the user.

In addition, the camera 14 and the microphone 16 may not be included in the information processing system, and at least one may be included in the information processing system. Environment information measuring devices other than these may be included in the information processing system. For example, a temperature sensor that measures temperature, a humidity sensor that measures humidity, an odor sensor that measures odor, an illuminance sensor that measures brightness, an infrared sensor, and the like may be included in the information processing system. Data measured by these sensors corresponds to an example of environment information. One or a plurality of environment information measuring devices may be included in the information processing apparatus 10. For example, the camera 14 may be included in the information processing apparatus 10. For example, the microphone 16 may be included in the information processing apparatus 10.

The device 18 is, for example, a PC, a tablet PC, a smartphone, a mobile phone, a robot (for example, a humanoid robot, a non-human animal robot, a cleaning robot, and other robots), a projector, a display device such as a liquid crystal display, a recording device, a playback device, an imaging device such as a camera, a refrigerator, a rice cooker, a microwave oven, a coffee maker, a vacuum cleaner, a washing machine, an air conditioner, a lighting device, a watch, a surveillance camera, an automobile, a motorcycle, aircraft (for example, unmanned aircraft (for example, drone)), a game machine, a gas range, a warm water washing toilet seat, a ventilation fan, a doorbell, an entrance monitor, an elevator, a door, a window, or various sensing devices (for example, a temperature sensor, a humidity sensor, a voltage sensor, a current sensor, or the like). The category of the concept of the device 18 may include general devices. For example, information device, video device, audio device, and other devices may also be included in the category of the device 18 according to the present exemplary embodiment.

The device 18 includes a communication device that is a communication interface, a storage device that stores data, and a processor that controls the operation of the device 18. The device 18 may have a user interface. The device 18 may transmit device identification information for identifying the device 18 that is the device 18 to the information processing apparatus 10. The device identification information is, for example, the ID, name, model number, or address (for example, MAC address or IP address) of the device 18.

Hereinafter, the configuration of the information processing apparatus 10 will be described in detail.

The information processing apparatus 10 includes, for example, a communication device 20, a UI 22, a storage device 24, and a processor 26. The information processing apparatus 10 may include other configurations.

The communication device 20 is a communication interface, and has a function of transmitting data to other apparatuses and a function of receiving data transmitted from other apparatuses. The communication device 20 may have a wireless communication function or may have a wired communication function.

The communication device 20 may communicate with other devices by using, for example, near field communication, or may communicate with other devices through a communication path such as a LAN or the Internet. For example, the communication device 20 receives the biological information transmitted from the biological information measuring device. The communication device 20 may transmit control information for controlling the operation of the biological information measuring device 12 to the biological information measuring device 12. Further, the communication device 20 receives the environment information transmitted from the environment information measuring device 12. The communication device 20 may transmit control information for controlling the operation of the environment information measuring device to the environment information measuring device. Further, the communication device 20 transmits control information for controlling the operation of the device 18 to the device 18. The communication device 20 may receive information transmitted from the device 18.

The UI 22 is a user interface, and includes a display device and an operation device. The display device is a liquid crystal display, an EL display, or the like. The operation device is a keyboard, input keys, an operation panel, or the like. The UI 22 may be a UI such as a touch panel that has both a display device and an operation device. In addition, a microphone may be included in the UI 22, and a speaker that emits sound may be included in the UI 22.

The storage device 24 is a device that constitutes one or a plurality of storage areas for storing various types of data. The storage device 24 is, for example, a hard disk drive, various memories (for example, RAM, DRAM, ROM, or the like), other storage devices (for example, an optical disk), or a combination thereof. One or a plurality of storage devices 24 are included in the information processing apparatus 10.

The storage device 24 stores management information. The management information is information for managing information for operating a device which implements a means for coping with a state of the user.

The category of the concept of the user state includes, for example, the user's emotion, mental state, psychological state, desire, physiological state, state related to the body (for example, state related to body function and organization), and the like. The state of the user is recognized based on the biological information measured from the user.

The means for coping with the state of a user includes, for example, means for improving the state of the user, means for satisfying the user's desire, means for solving the problem of the user, or the like, and is determined in advance. Hereinafter, means for coping with the state of the user will be referred to as “coping means”.

For example, the user state information indicating the user state, the reference biological information assumed to be generated from the user having the state, the coping means information indicating the coping means for coping with the state, and the device information for identifying a device which implements the coping means are registered in advance in the management information in association with each other. For each user, user state information, reference biological information, coping means information, and device information may be associated with each other and registered in the management information.

The processor 26 is configured to operate the device 18 based on the biological information of the user. For example, the processor 26 receives biological information of the user, and outputs, to a device 18 which implements the coping means for coping with the state of the user recognized from the biological information, an operation instruction for implementing the coping means.

For example, a brain machine interface may be constructed by the biological information measuring device 12 that measures brain activity and the information processing apparatus 10. The brain machine interface method may be invasive or non-invasive. In this case, the processor 26 operates the device 18 based on the user's brain activity (for example, brain waves). In order to operate the device 18, the processor 26 may extract a characteristic component from the brain wave, and operate the device 18 based on the extracted component. In order to extract characteristic components from the brain wave, Fast Fourier Transform (FFT), Wavelet Transform (WT), Time Frequency Distribution (TFD), Eigenvector Methods (EM), autoregressive model (ARM) or the like may be used. In addition, as a method for linking the brain wave and the operation of the device 18 using the feature vector obtained by the feature extraction, for example, independent component analysis (ICA), k-average method, support vector machine (SVM), a convolutional neural network, or the like may be used.

Further, the processor 26 is configured to receive the device identification information transmitted from the device 18 and identify the device 18. For example, the processor 26 transmits a device identification information acquisition request to the device 18 and acquires the device identification information transmitted from the device 18 in response to the acquisition request. Further, device identification information is transmitted to the information processing apparatus 10 from the device 18 that can communicate with the information processing apparatus 10 by being connected to the information processing apparatus 10, and the processor 26 may receive the device identification information that has been transmitted in this manner.

The processor 26 is configured to control the operation of each unit of the information processing apparatus 10. The processor 26 may include a memory.

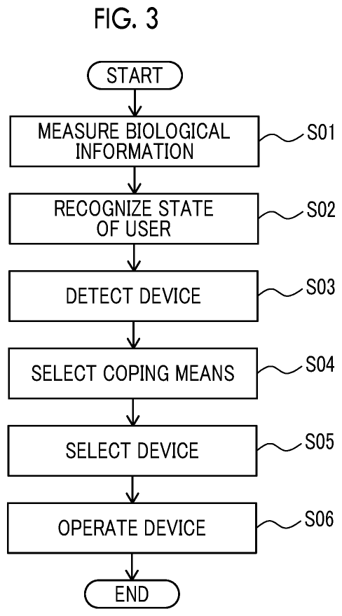

Hereinafter, the processing by the information processing apparatus 10 will be described in detail with reference to FIG. 3.

The biological information of the user is measured by the one or plurality of biological information measuring devices 12 (step S01). The measured biological information is transmitted to the information processing apparatus 10.

The processor 26 receives the biological information measured from the user and recognizes the state of the user based on the biological information (step S02).

For example, the processor 26 compares the biological information measured from the user with each piece of reference biological information registered in the management information described above, and searches for reference biological information whose difference from the biological information measured from the user is included in the allowable range. The processor 26 estimates that the state indicated by the user state information associated with the searched reference biological information is the state of the user whose biological information has been measured. In a case where a plurality of pieces of reference biological information whose difference from the biological information measured from the user is included in the allowable range are searched, the processor 26 specifies reference biological information whose difference from the biological information measured from the user is minimum, from among the plurality of pieces of reference biological information. The processor 26 estimates that the state indicated by the user state information associated with the specified reference biological information is the state of the user whose biological information has been measured. The allowable range is predetermined. The allowable range may be changed by the user.

The reference biological information may be information indicating characteristic components of the biological information. In this case, the processor 26 may extract a characteristic component from the biological information measured from the user, and search for reference biological information whose difference from the extracted component is within an allowable range. The processor 26 estimates that the state indicated by the user state information associated with the searched reference biological information is the state of the user whose biological information has been measured.

For example, in a case where a brain wave is used as the biological information, the processor 26 may estimate the state of the user whose brain wave is measured, by extracting a characteristic component from the measured brain wave and analyzing the component.

Next, the processor 26 detects the user whose biological information has been measured, or one or a plurality of devices 18 that can act on the surroundings of the user (step S03). For example, the processor 26 detects the devices 18 acting on the user or the surroundings of the user, based on the relationship between the position of the user whose biological information is measured and the position of each device 18. The surroundings of the user includes the position of the user and is a range which is predetermined with the position of the user as a reference.

Next, the processor 26 selects coping means for coping with the state of the user (step S04). For example, the processor 26 searches for the coping means information associated with the user state information indicating the state specified in step S02 in the management information described above, and selects the coping means indicated by the searched coping means information as a coping means for coping with the state of the user.

Next, the processor 26 selects the device 18 which implements the coping means selected in step S04 (step S05). The processor 26 selects devices 18 which implements the coping means selected in step S04, from one or a plurality of devices 18 which are detected in step S03 and capable of acting on the user or the surroundings of the user. For example, the processor 26 selects devices 18 which are indicated by the device information associated with the coping means information indicating the coping means selected in step S04 in the management information described above, and capable of acting on the user or the surroundings of the user.

Next, the processor 26 operates the device 18 by transmitting control information indicating an operation instruction for implementing the coping means selected in step S04 to the device 18 selected in step S05 (step S06). The device 18 that has received the control information operates according to the control information, thereby implementing the coping means selected in step S04. As described above, the device 18 which implements the coping means for coping with the state of the user is selected, and an operation instruction can be output to the device.

Hereinafter, the operation of the information processing system according to the present exemplary embodiment will be described with a specific example.

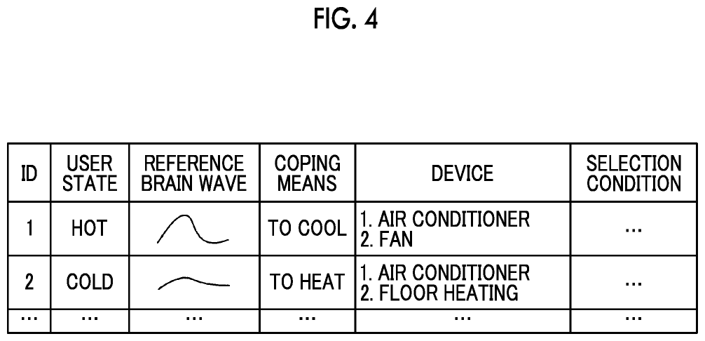

FIG. 4 shows an example of a management table that is an example of management information. Data of the management table is stored in the storage device 24. The data of the management table may be stored in an external device such as a server without being stored in the storage device 24.

In the management table, the ID, the user state information indicating the user state, the reference brain wave assumed to be generated from the user having the state, the coping means information indicating the coping means for coping with the state, the device information for identifying a device which implements the coping means, and selection condition information indicating a selection condition for selecting the device are associated with each other. The reference brain wave is an example of reference biological information. Here, brain waves are used as an example of biological information, but biological information other than brain waves may be used.

The ID is information for managing information registered in the management table. The reference brain wave is, for example, a brain wave that is determined by statistical processing and is assumed to be generally generated from a user who has a state associated with the reference brain wave. The device information is, for example, the name, device ID, or model number of the device 18.

Hereinafter, each piece of information associated with the ID “1” will be described in detail.

The state indicated by the user state information associated with the ID “1” is a state in which the user feels “hot”. The reference brain wave associated with the ID “1” is a brain wave that is assumed to be generated from the user when the user feels “hot”. That is, in a case where a brain wave whose difference from the reference brain wave associated with the ID “1” is within an allowable range is measured from the user, it is estimated that the user feels “hot”. The coping means information associated with the ID “1” is information indicating coping means for cooling the surroundings of the user in order to cope with the state of the user who feels “hot”. The coping means “to cool” is means for improving the state of the user who feels “hot”. Specifically, the coping means “to cool” is means for preventing a user who feels “hot” from feeling “hot”. As a device 18 which implements the coping means “to cool”, “air conditioner” and “electric fan” are associated with the coping means “to cool”. That is, “air conditioner” and “electric fan” are registered in the management table as devices 18 which actually implement the coping means “to cool”. In the example shown in FIG. 4, two devices 18 are associated with the coping means, but one or a plurality of devices 18 which implement the coping means may be associated with the coping means. The selection condition information associated with the ID “1” is information indicating a condition for selecting the device 18 to be actually operated, among the “air conditioner” and “fan” that are the devices 18. Examples of the selection conditions include conditions related to the environment around the user and the device 18, the position of the user, the position of the device 18, the state of the user, the performance of the device 18, or the extent to which the device 18 acts on the user and the surroundings of the user.

For example, in a case where a brain wave whose difference from the reference brain wave associated with the ID “1” is within an allowable range is measured from the user, the processor 26 estimates that the user feels “hot”. For example, the processor 26 extracts features such as the shape, peak, period, and amplitude of the brain wave from each of the brain wave measured from the user and the reference brain wave, compares the features of waveforms, and determines whether or not a difference between the features of both waveforms is included in the allowable range.

Further, the processor 26 may calculate the similarity between the brain wave measured from the user and the reference brain wave, and may determine whether or not the similarity is equal to or greater than a threshold. The threshold is a value corresponding to the allowable range. In a case where the similarity between the two waveforms is equal to or greater than the threshold, the processor 26 determines that the two waveforms are similar, and determines that the difference between the waveform measured from the user and the reference brain wave is included in an allowable range. That is, in a case where a brain wave whose similarity with the reference brain wave associated with the ID “1” is equal to or greater than the threshold is measured from the user, the processor 26 estimates that the user feels “hot”.

Note that the user state information may be associated with a plurality of pieces of different reference biological information. For example, the user state information indicating the state of the user of “hot” may be associated with a reference brain wave assumed to be measured from a user who feels “hot”, and a reference body temperature assumed to be the user's body temperature at that time as reference biological information. In this case, in a case where the difference between the brain wave measured from the user and the reference brain wave is included in the allowable range, and the difference between the user's body temperature measured at that time and the reference body temperature is included in the allowable range, the processor 26 may estimate that the user feels “hot”.

The processor 26 operates the device 18 by selecting the device 18 that actually implements the coping means according to the selection condition, and transmitting control information indicating an operation instruction to the device 18. For example, the processor 26 selects “air conditioner” according to the selection condition, and in order to implement the coping means “to cool”, and turns the cooling of the “air conditioner” on by transmitting control information indicating an operation instruction to turn on the cooling of the “air conditioner” to the “air conditioner”.

Note that the output level of the device 18 according to the state of the user may be associated with the coping means and the device 18 and registered in the management table. For example, the set temperature and air volume about the cooling of the “air conditioner” that is the device 18 may be associated with the coping means and the “air conditioner”. The set temperature and the air volume are determined according to the state of the user. In this case, the processor 26 transmits control information including the set temperature and the air volume to the “air conditioner”, thereby setting the set temperature and the air volume of the “air conditioner” in the “air conditioner”.

For each user, user state information, reference biological information, coping means information, device information, and selection condition information may be associated with each other and registered in the management table. For example, biological information measured from a user may be registered in the management table as the reference biological information of the user.

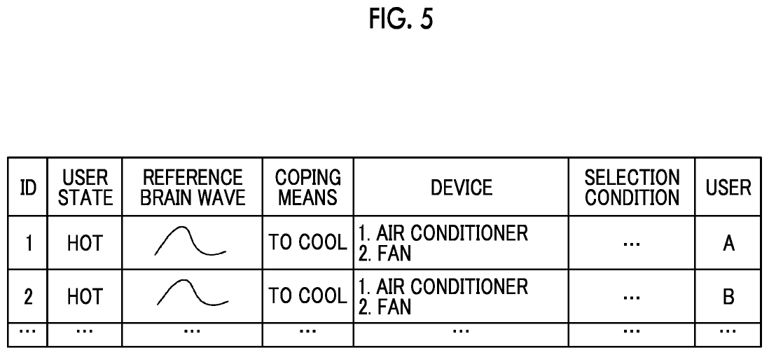

FIG. 5 shows a management table in which specific reference biological information of each user is registered. In the management table shown in FIG. 5, an ID, user state information, a reference brain wave which is an example of reference biological information, coping means information, device information, selection condition information, and user information are associated with each other. User information is information (for example, a user name, a user ID, or the like) for identifying a user.

The reference brain wave associated with the user information is a brain wave measured from the user when the user indicated by the user information has a state indicated by the user state information associated with the reference brain wave. A reference brain wave representing each state of each user is measured in advance and registered in the management table.

For example, a brain wave of the user A when the user A feels “hot” is measured by the biological information measuring device 12, and the measured brain wave is registered as a reference brain wave generated when the user A feels “hot” in the management table in association with the user state information indicating the state of “hot”. Further, coping means information, device information, selection condition information, and user information indicating the user A are registered in the management table in association with the user state information and the reference brain wave. This registration work may be performed using the information processing apparatus 10 or may be performed using another apparatus. In the example shown in FIG. 5, these pieces of information are registered as information of ID “1”. Similarly, each piece of information is registered in the management table for each of other states or other users.

The registration work may be performed a plurality of times, and an average of a plurality of brain waves measured thereby may be registered as a reference brain wave. For example, a work of measuring the brain wave of the user A when the user A feels “hot” by the biological information measuring device 12 is performed a plurality of times, and the average of a plurality of brain waves measured by this work may be registered in the management table as a reference brain wave.

For example, in a state where only the user A logs in to the information processing apparatus 10, in a case where a brain wave having the difference from the reference brain wave associated with the ID “1” being within an allowable range is measured from the user A, the processor 26 estimates that the user A feels “hot”. Then, the processor 26 selects the coping means “to cool” as the coping means for coping with the state of the user A, and specifies the device 18 which implements the coping means. The processor 26 selects the device 18 which actually implements the coping means “to cool”, according to the selection condition, and operates the selected device 18.

As another example, in the state where a fact that the user operating the device 18 is “user A” is set in the information processing apparatus 10, in a case where a brain wave having the difference from the reference brain wave associated with the ID “1” being within an allowable range is measured from the user A, the processor 26 may estimate that the user A feels “hot”.

As for the user other than the user A, similar to the user A, each information is registered in the management table. For example, each piece of information associated with the ID “2” is information indicating coping means when the user B feels “hot”.

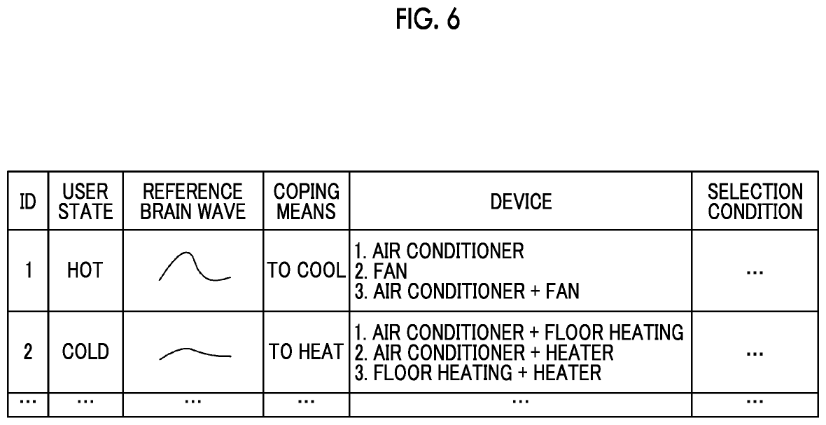

The coping means may be implemented by a single device 18 or the coping means may be implemented by a combination of a plurality of devices 18. For example, as shown in FIG. 6, a combination of a plurality of devices 18 which implement the coping means may be registered in the management table. To explain with a specific example, “air conditioner” that is a single device 18, “fan” that is a single device 18, and “air conditioner+fan” that is a combination of the devices 18 are associated with the coping means of ID “1”. That is, the coping means “to cool” may be implemented by “air conditioner” or “fan” that is a single device 18, or may be implemented by a combination of “air conditioner” and “fan”. The processor 26 selects the device 18 that actually implements the coping means, from among the “air conditioner”, “fan”, or “air conditioner+fan”, according to the selection condition. Another coping means is associated with a single device 18 which implements the other coping means or a combination of the devices 18.

Hereinafter, processing for selecting the device 18 that actually implements the coping means according to the selection condition will be described.

For example, the processor 26 may select the devices 18 acting on the user or the surroundings of the user as the devices 18 that actually implement the coping means, from among one or a plurality of devices 18 associated with the coping means. The processor 26 operates the device 18 by transmitting control information indicating an operation instruction to the selected device 18. For example, the processor 26 specifies the position of the user and the position of each device 18 associated with the coping means, and selects the device 18 capable of acting on the user or the surroundings of the user as the device 18 that actually implements the coping means, based on the relationship between the position of the user and the position of each device 18. For example, in a case where the coping means is “to cool”, the processor 26 selects the device 18 capable of sending wind to the user or the surroundings of the user as the devices 18 that actually implement the coping means. In a case where the “air conditioner” is the device 18 capable of sending wind to the user or the surroundings of the user, and the “fan” is a device 18 that is not capable of sending wind to the user or the surroundings of the user, the processor 26 selects “air conditioner” as the device 18 that actually implements the coping means, and transmits the control information to “air conditioner”, thereby turning on the cooling of “air conditioner”. That is, in a case where the “air conditioner” is installed in the position where the air sent from the “air conditioner” reaches the user or the surroundings of the user, and the “fan” is installed in the position where the air sent from the “fan” does not reach the user or the surroundings of the user, the processor 26 turns on the cooling of the “air conditioner”. The processor 26 specifies the position of the user and the position of the device 18 by using a global positioning system (GPS). In addition, the position of the user is the position of the terminal device (for example, a smart phone, a mobile phone, or the like) that the user is carrying, for example. The user and each device 18 are captured by the camera 14, and the processor 26 may recognize the position relationship between the user and each device 18 by analyzing image data generated by the capturing.

The processor 26 may select the devices 18 having the greatest effect on coping with the state of the user as the devices 18 that actually implement the coping means, from among one or a plurality of devices 18 associated with the coping means. In a case where the description will be made using the above example, the processor 26 selects an air conditioner capable of sending the strongest wind to the user or the user's surroundings as a device 18 that actually implements coping means, among a plurality of air conditioners.

The processor 26 may select a device 18 corresponding to the conversation contents of the user when the biological information representing the state of the user registered in the management table is measured as a device 18 that actually implements the coping means, from among one or a plurality of devices 18 associated with the coping means. The conversation contents are conversation contents when the biological information is measured. For example, the conversation between the user and the conversation partner is measured by the microphone 16, and the processor 26 analyzes the conversation contents to recognize the environment where the user is located, and selects the device 18 suitable for the environment as the device 18 that actually implements the coping means. For example, the processor 26 counts the number of people who are talking from the conversation contents, and selects the device 18 corresponding to the number of people as the device 18 that actually implements the coping means.

The processor 26 may select the device 18 corresponding to the conversation partner of the user as the device 18 that actually implements the coping means, from among one or a plurality of devices 18 associated with the coping means.

The processor 26 may select a device 18 corresponding to the work contents of the user when the biological information representing the state of the user registered in the management table is measured as a device 18 that actually implements the coping means, from among one or a plurality of devices 18 associated with the coping means. The work contents are work contents when the biological information is measured. For example, the user's operation is captured by the camera 14, and the processor 26 analyzes the image data generated by the capturing so as to recognize the work contents of the user and select the device 18 suitable for the work contents as the device 18 that actually implements the coping means.

The processor 26 may select the devices 18 according to the relationship between the position of the user and the position of each device 18 as the devices 18 that actually implement the coping means, from among one or a plurality of devices 18 associated with the coping means. For example, the processor 26 selects the device 18 that is installed at a position closest to the user's position as the devices 18 that actually implement the coping means, from among one or a plurality of devices 18 associated with the coping means.

The processor 26 may select the devices 18 having a higher search ranking on the Internet as the devices 18 that actually implement the coping means, from among one or a plurality of devices 18 associated with the coping means.

The processor 26 may select the newest device 18 as the devices 18 that actually implement the coping means, from among one or a plurality of devices 18 associated with the coping means. The newest device 18 may be, for example, the device 18 with the newest manufacturing date, or may be the device 18 with the newest installation date.

Hereinafter, specific examples will be described in detail.

### Example 1

In Example 1, the processor 26 selects the device 18 that actually implements the coping means, based on the relationship between the position of the user and the position of each device 18.

For example, in a case where a brain wave whose difference from the reference brain wave representing the user state “I want to watch TV” is included in the allowable range is measured from the user, the processor 26 specifies one or a plurality of devices 18 associated with the user state “I want to watch TV” in the management table. For example, “television” is registered in the management table as a device 18 for coping with the user state “I want to watch TV”. In this case, the processor 26 transmits control information indicating an operation instruction to turn on the power of the “television” to the “television” installed at the position closest to the user's position, thereby turning on the power of the “television”.

### Example 2

In Example 2, the processor 26 selects the device 18 that most acts on the user or the surroundings of the user as the device 18 that actually implements the coping means. The device 18 that most acts is, for example, the device 18 that is installed at the most effective position with respect to the user or the surroundings of the user.

For example, in a case where a brain wave whose difference from the reference brain wave representing the user state “sultry” is included in the allowable range is measured from the user, the processor 26 specifies one or a plurality of devices 18 associated with the user state “sultry” in the management table. For example, “air conditioner” and “dehumidifier” are registered in the management table as devices 18 for coping with the state of the user “sultry”. In this case, the processor 26 selects the device 18 installed at the most effective position with respect to the user or the surroundings of the user, from among “air conditioner” and “dehumidifier”. For example, in a case where the wind sent from the “air conditioner” reaches the user or the user's surroundings and the user or the user's surroundings are not included in the area where the dehumidifying effect of the “dehumidifier” is obtained, the processor 26 selects “air conditioner” as the device 18 installed in the most effective position, and transmits control information indicating a dehumidifying operation instruction to the “air conditioner”, thereby turning on the dehumidifying function of the “air conditioner”.

### Example 3

In Example 3, the processor 26 operates the plurality of devices 18 by selecting a combination of the plurality of device 18 which implement the coping means and transmitting control information to the plurality of devices 18.

For example, in a case where a brain wave whose difference from the reference brain wave representing the user state “cold” is included in the allowable range is measured from the user, the processor 26 specifies a combination of a plurality of devices 18 associated with the user state “cold” in the management table. For example, a combination of “floor heating”, “air conditioner”, and “warm air heater” is registered in the management table as a combination of a plurality of devices 18 for coping with the state of the user “cold”. In addition, each of “floor heating”, “air conditioner”, and “warm air heater” may be registered in the management table as a single device 18 for coping with the state of the user “cold”. For example, in a case where it is possible to heat the user or the surroundings of the user the fastest by using the combination of “floor heating”, “air conditioner”, and “warm air heater”, the processor 26 selects the combination as a combination of the devices 18 that actually implement coping means. The processor 26 transmits control information indicating a heating operation instruction to each of “floor heating”, “air conditioner”, and “warm air heater”, thereby turning on the heating functions of “floor heating”, “air conditioner”, and “warm air heater”.

### Example 4

In Example 4, the processor 26 selects a device 18 corresponding to the work contents of the user when the biological information representing the state of the user registered in the management table is measured as a device 18 that actually implements the coping means, from among one or a plurality of devices 18 associated with the coping means. The work contents are work contents when the biological information is measured.

For example, in a case where the user is working while sitting at a desk, the state of the work is captured by the camera 14. The processor 26 recognizes the work contents of the user by analyzing the image data generated by the capturing. For example, in a case where the user is reading a document, the processor 26 transmits control information indicating an operation instruction to turn on the lighting to the lighting device that illuminates the user's hand. Thereby, a user's hand is illuminated by the lighting device. As another example, when the user is sitting at a desk, in a case where a brain wave whose difference from the reference brain wave representing the psychological state of visual information processing “reading a document” is within an allowable range is measured from the user, the processor 26 transmits control information indicating an operation instruction to turn on the lighting to the lighting device that illuminates the user's hand. Thereby, a user's hand is illuminated by the lighting device. In a case where the user creates a document using a PC, the processor 26 transmits control information indicating an operation instruction to turn off the lighting to the lighting device that illuminates the user's hand, and transmits control information indicating an operation instruction to turn on the lighting to the lighting device installed on the ceiling. Thereby, the whole room is illuminated by the lighting device installed on the ceiling. As another example, when the user is sitting at a desk, in a case where a brain wave whose difference from the reference brain wave representing a creative psychological state of “creating data using a PC” is within an allowable range is measured from the user, the processor 26 may transmit control information indicating an operation instruction to turn off the lighting to the lighting device that illuminates the user's hand, and transmit control information indicating an operation instruction to turn on the lighting, to the lighting device installed on the ceiling.

### Example 5

In Example 5, the processor 26 selects the device 18 corresponding to the conversation contents of the user as the device 18 that actually implements the coping means. More specifically, the processor 26 selects the device 18 corresponding to the conversation partner of the user as the device 18 that actually implements the coping means.

For example, in a case where a brain wave whose difference from the reference brain wave representing a request “I want to operate lighting” is included in the allowable range is measured from the user, the processor 26 specifies one or a plurality of devices 18 associated with the user state “I want to operate lighting” in the management table. For example, “lighting device installed on the ceiling” and “indirect lighting device” are registered in the management table as the devices 18 for coping with the user state “I want to operate lighting”. In this case, the processor 26 selects a lighting device according to the conversation partner of the user, from among “lighting device installed on the ceiling” and “indirect lighting device”. For example, in a case where a family including the user are talking with each other or in a case where the user is talking with a friend, the processor 26 transmits control information indicating an operation instruction to turn on the lighting to the “lighting device installed on the ceiling”. Thereby, the room is illuminated by the “lighting device installed on the ceiling”. In a case where the user is talking to the lover, the processor 26 transmits control information indicating an operation instruction to turn on the lighting to the “indirect lighting device”.

### Example 6

In Example 6, the processor 26 selects the device 18 corresponding to the conversation contents of the user as the device 18 that actually implements the coping means.

For example, a plurality of people including the user have a conference, and the state is captured by the camera 14. The processor 26 recognizes that a conference is being held by a plurality of people, by analyzing the image data generated by the capturing. Further, in a case where a brain wave whose difference from the reference brain wave representing a request “I want to display presentation materials” is included in the allowable range is measured from the user, the processor 26 specifies one or a plurality of devices 18 associated with the user state “I want to display presentation materials” in the management table. For example, “liquid crystal display” and “projector” are registered in the management table as a device 18 for coping with a user state “I want to display presentation materials”. The condition for selecting “liquid crystal display” as the device 18 which actually implements the coping means is that the number of participants in the conference is not more than a threshold (for example, not more than three). The condition for selecting “projector” as the device 18 which actually implements the coping means is that the number of participants in the conference exceeds the threshold. These conditions are registered in the management table as selection conditions. The processor 26 may estimate the number of participants in the conference by analyzing the image data generated by the capturing by the camera 14, or estimate the number of participants in the conference by analyzing the voice data generated by the measurement by the microphone 16.

In a case where the number of participants in the conference is not more than the threshold, the processor 26 transmits control information indicating an operation instruction to turn on the power of the “liquid crystal display” to turn on the power of the “liquid crystal display”. In a case where the number of participants in the conference exceeds the threshold, the processor 26 transmits control information indicating an operation instruction, thereby turning on the power of the “projector” to turn on the power of the “projector”.

In the embodiments above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device). In the embodiments above, the term “processor” is broad enough to encompass one processor or plural processors in collaboration which are located physically apart from each other but may work cooperatively. The order of operations of the processor is not limited to one described in the embodiments above, and may be changed.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing apparatus comprising:
a processor configured to:
receive biological information of a user, and
output, to a device which implements means for coping with a state of the user recognized from the biological information, an operation instruction for implementing the means.

2. The information processing apparatus according to claim 1,
wherein the processor further receives information from the device and identifies the device.

3. The information processing apparatus according to claim 1,
wherein the device acts on the user or surroundings of the user.

4. The information processing apparatus according to claim 2,
wherein the device acts on the user or surroundings of the user.

5. The information processing apparatus according to claim 1,
wherein the processor outputs the operation instruction to a device according to conversation contents of the user, the conversation contents are conversation contents when the biological information is measured.

6. The information processing apparatus according to claim 2,
wherein the processor outputs the operation instruction to a device according to conversation contents of the user, the conversation contents are conversation contents when the biological information is measured.

7. The information processing apparatus according to claim 3,
wherein the processor outputs the operation instruction to a device according to conversation contents of the user, the conversation contents are conversation contents when the biological information is measured.

8. The information processing apparatus according to claim 4,
wherein the processor outputs the operation instruction to a device according to conversation contents of the user, the conversation contents are conversation contents when the biological information is measured.

9. The information processing apparatus according to claim 1,
wherein the processor outputs the operation instruction to a device according to work contents of the user, the work contents are work contents when the biological information is measured.

10. The information processing apparatus according to claim 2,
wherein the processor outputs the operation instruction to a device according to work contents of the user, the work contents are work contents when the biological information is measured.

11. The information processing apparatus according to claim 3,
wherein the processor outputs the operation instruction to a device according to work contents of the user, the work contents are work contents when the biological information is measured.

12. The information processing apparatus according to claim 4,
wherein the processor outputs the operation instruction to a device according to work contents of the user, the work contents are work contents when the biological information is measured.

13. The information processing apparatus according to claim 1,
wherein the state of the user is emotion of the user, and
wherein the processor outputs the operation instruction to a device according to the emotion of the user.

14. The information processing apparatus according to claim 2,
wherein the state of the user is emotion of the user, and
wherein the processor outputs the operation instruction to a device according to the emotion of the user.

15. The information processing apparatus according to claim 3,
wherein the state of the user is emotion of the user, and
wherein the processor outputs the operation instruction to a device according to the emotion of the user.

16. The information processing apparatus according to claim 1,
wherein the processor outputs the operation instruction to a device according to a relationship between a position of the user and a position of the device.

17. The information processing apparatus according to claim 16,
wherein the processor outputs the operation instruction to a device which implements the means and is installed at a position closest to the position of the user.

18. The information processing apparatus according to claim 1,
wherein the processor outputs the operation instruction to a device having a greatest effect on the state of the user.

19. The information processing apparatus according to claim 1,
wherein the processor outputs the operation instruction to a device corresponding to a conversation partner of the user.

20. A non-transitory computer readable medium storing a program causing a computer to:
receive biological information of a user, and
output, to a device which implements means for coping with a state of the user recognized from the biological information, an operation instruction for implementing the means.
